(19) United States
(12) Patent Application Publication
(10) Pub. No.: US 2009/0062831 A1
(43) Pub. Date: Mar. 5, 2009

(54) GUIDE DEVICE FOR AN ACUPUNCTURE NEEDLE

(76) Inventors: Klaus Teichert, Pullach (DE); Elmar Grandy, Oberschleissheim (DE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 12/280,135

(22) PCT Filed: Feb. 20, 2007
(86) PCT No.: PCT/EP07/01453

§ 371 Date: Aug. 20, 2008

(30) Foreign Application Priority Data
Feb. 20, 2006 (DE) 10 2006 007 803.9

(51) Int. Cl. A61B 17/34

(52) U.S. Cl. 606/189

(57) ABSTRACT

A guide device (100), in particular for an acupuncture needle (1), includes a guide tube (10) for accommodating the acupuncture needle (1), with a needle part (3) and a needle handle (2), and a clamping device (20) for releasably fixing the acupuncture needle (1) to the guide tube (10), wherein the clamping device (20) has at least one clamping element (21) which is movable with respect to the guide tube (10).

The invention relates to a guide device, in particular for an acupuncture needle, with a guide tube and a clamping device for releasably fixing the acupuncture needle to the guide tube. The invention also relates to an acupuncture device which is equipped with such a guide device, and to a method for releasing an acupuncture needle using such a guide device.

In order to stimulate an organism, an acupuncture needle is typically oriented firstly in conjunction with a guide tube (or guide pipe) towards the surface of a body part that is to be stimulated, and is then introduced into the body part. Various concepts for releasably connecting the acupuncture needle and the guide tube are known from practice. By way of example, an adhesive bond may be formed, but this may have disadvantages with regard to assembly and the strength of the bond. Furthermore, a protrusion may be provided in the interior of the guide tube, which protrusion engages in a groove in the handle of the acupuncture needle. In this case, disadvantages result due to the limitation that only certain acupuncture needles can be combined with a suitable guide tube.

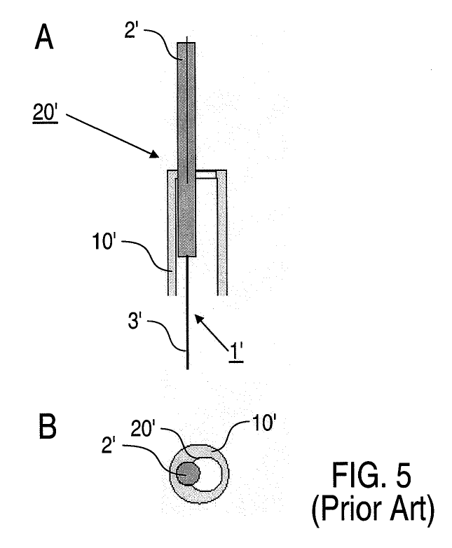

US 2005/00021067 A1 describes a guide tube 10′ for an acupuncture needle 1′ with a needle handle 2′ and a needle part 2′, which is equipped with a clamping device 20′. The mode of action of the conventional clamping device 20′ is illustrated in FIG. 5A (longitudinal section) and 5B (cross section). The clamping device 20′ comprises at the end of the guide tube 10′ for accommodating the needle handle 2′ of the acupuncture needle 1′ a clamping structure which has a narrow opening for the releasable fixing of the needle handle 2′ and a wide opening for the free movement of the needle handle 2′. In order to release the acupuncture needle 1′, the needle handle 2′ is displaced radially from the narrow to the wide opening. The technique according to US 2005/00021067 A1 has the disadvantage that the release movement of the acupuncture needle 1′ differs from the axial forward movement direction of the acupuncture needle 1′ when being introduced into the organism and requires a relatively large amount of force, so that the placement of the acupuncture needle 1′ takes a lot of time and is made more difficult.

The objective of the invention is to provide an improved guide device for acupuncture needles, by means of which the disadvantages of the conventional guide devices are overcome and which in particular allows reliable and stable protection of the acupuncture needle and the rapid and low-force release of the acupuncture needle for the placement of the latter. The objective of the invention is also to provide a correspondingly improved acupuncture device and an improved method for releasing acupuncture needles.

These objectives are achieved by devices or methods having the features of claims 1, 19 and 21. Advantageous embodiments and applications of the invention emerge from the dependent claims.

According to a first aspect, the objective of the invention is achieved by the general technical teaching of providing a guide device for an acupuncture needle comprising a guide tube and a clamping device, which has at least one movable clamping element. The guide tube is an elongate, straight component with a hollow tubular or hose shape, on or in which the acupuncture needle can be releasably fixed. The guide tube is a casing in particular for a needle part of the acupuncture needle. The clamping device is designed to form a seat preferably for a needle handle of the acupuncture needle. The at least one movable clamping element can be transferred between a clamped state, in which the acupuncture needle can be fixed to the guide tube, and a released state, in which the acupuncture needle is released and can be separated from the guide tube. The clamping device can be transferred to the released state by means of a force which is exerted on the at least one movable clamping element and is directed axially or radially relative to the longitudinal dimension of the guide tube.

By means of the at least one movable clamping element, the seat for the acupuncture needle is created for example in conjunction with a further clamping element or another part of the clamping device, whereby advantageously a high level of stability and strength of the fixing of the acupuncture needle and flexibility in terms of adaptation to different types of acupuncture needles is achieved. It is particularly advantageous that the acupuncture needle can be released by a movement of the at least one clamping element relative to the guide tube, with said acupuncture needle not being displaced or being displaced substantially axially.

The mobility of the clamping element comprises the ability of the clamping element to be displaced or pivoted relative to the guide tube. According to a preferred embodiment of the invention, the at least one clamping element comprises a flexible material. The clamping element is preferably formed in an elastically deformable or springy manner. Advantageously, a springy clamping element adapts easily to different types of acupuncture needles, e.g. with a plastic or metal needle handle or a coiled wire needle handle.

If the at least one clamping element forms a holder for the axially centered positioning of the acupuncture needle, in particular for the needle part in the guide tube, advantages may be obtained with regard to the alignment of the guide tube with the acupuncture needle with respect to an organism that is to be stimulated.

According to another preferred embodiment of the invention, the clamping device is connected to an end of the guide tube. Advantageously, the needle part of the acupuncture needle can be accommodated fully in the guide tube when the acupuncture needle is clamped at the needle handle. If, according to a first variant, the clamping device is connected in one piece with the guide tube, advantages are obtained with regard to the production of the guide device, e.g. in the plastic injection molding method. The clamping device is optionally formed by one end of the guide tube. If, according to a second variant, the clamping device is releasably fixed to the guide tube, advantages may be obtained in terms of adapting to different types of needle and an increased functionality of the guide device.

If, according to a particularly preferred embodiment of the invention, the clamping device forms a sleeve, advantages may be obtained with regard to a stable and centered fixing of an acupuncture needle in the guide tube. The term “sleeve” refers here in general to a structure which radially surrounds the seat for the acupuncture needle. An interior of the sleeve is widened or narrowed by the movement of the at least one clamping element. The sleeve has a circumferential wall or a plurality of sleeve parts which are connected at a sleeve end, e.g. by a ring.

The clamping device in the form of a sleeve has particular advantages with regard to handling of the acupuncture device, particularly when releasing the acupuncture needle using just one hand. The acupuncture needle can be released by means of one single movement sequence.

According to a first variant, the sleeve is formed by at least two spring clamping elements, between which the acupuncture needle can be clamped. The spring clamping elements are resilient blades which are connected to one another at their ends. In a relaxed state, the spring clamping elements make contact with one another. In the clamped state of the clamping device, the spring clamping elements are mounted at a distance from one another by the needle handle of the acupuncture needle. In order to release the acupuncture needle, the distance is increased by exerting a pressure force on the connected ends of the spring clamping elements, until the needle handle can move. Preferably, the sleeve has a cross section in the shape of a rhombus, which is advantageously formed by four flat spring clamping elements and can be actuated easily by hand.

According to a further variant, the sleeve has profile clamping elements on its inner side, between which a seat is formed for the acupuncture needle. Advantageously, the distance between the profile clamping elements can easily be increased by widening the sleeve. The widening of the sleeve can be achieved by displacing the sleeve and the guide tube relative to one another. In the case of a sleeve with an inner profile for example, it may be provided that the sleeve is seated on one end of the guide tube in the clamped state and is pushed onto the guide tube for release purposes. As a result of being pushed on, the sleeve is spread open and the distance between the profile clamping elements is increased.

Alternatively, in the case of a sleeve with an outer profile, it may be provided that the sleeve is pressed into a narrowing preferably at the end of the guide tube in the clamped state, and for release purposes is pushed into the guide tube. In both cases, the release of the acupuncture needle can advantageously be brought about by a simple axial movement of the sleeve.

If, according to a further modification of the guide device according to the invention, the sleeve is closed at an end opposite the guide tube, advantages may be obtained with regard to protecting the acupuncture needle in the guide device. According to another advantageous feature of the invention, the guide tube may also be closed at an end opposite the clamping device. The guide device can thus form a sterile housing for the acupuncture needle.

According to an alternative embodiment of the invention, the clamping device may comprise two gripper clamping elements which form a tongs-like seat for the acupuncture needle. The gripper clamping elements are arranged at a distance which is smaller than the diameter of the needle handles of typically used acupuncture needles. At least one of the clamping elements is elastically deformable. The gripper clamping elements are preferably arranged on a support of the clamping device which can be pivoted radially with respect to the guide tube. The support extends axially at one end of the guide tube. If the support is formed in one piece with the guide tube and is connected to the latter for example via a hinge, advantages are obtained with regard to simple production of the guide device.

According to the invention, the support may have a protrusion, e.g. in the form of a spike, which can advantageously be used as a tool when releasing the unit consisting of the guide device and the acupuncture needle from a dispenser, such as e.g. a magazine as described in DE 103 50 720.5 or a cartridge.

According to a second, independent aspect, the objective of the invention is achieved by the general technical teaching of providing an acupuncture device which comprises the guide device according to the invention and an acupuncture needle. Preferably, the acupuncture needle has a needle handle which can be releasably connected to the guide tube by the clamping device of the guide device.

Another independent subject matter of the invention is a method for releasing an acupuncture needle using the guide device according to the invention or the acupuncture device according to the invention. The method according to the invention is characterized in that at least one clamping element of a clamping device provided on a guide tube is deformed in order to deform a seat in which the acupuncture needle, in particular the needle handle, is arranged. The deformation brings about a widening of the seat, so that the acupuncture needle is separated from the guide tube and can be introduced into a body part of an organism to be treated.

Preferably, the clamping device is deformed by a force acting radially with respect to the longitudinal direction of the guide tube. In this case, advantages are obtained with regard to easy manual release of the acupuncture needle using two fingers. Alternatively, the clamping device is deformed by a force acting axially with respect to the longitudinal direction of the guide tube. Advantageously, radial displacements of the acupuncture needle prior to placement in the organism to be stimulated are avoided in this case.

Further details and advantages of the invention will become clear from the description of the appended drawings, in which:

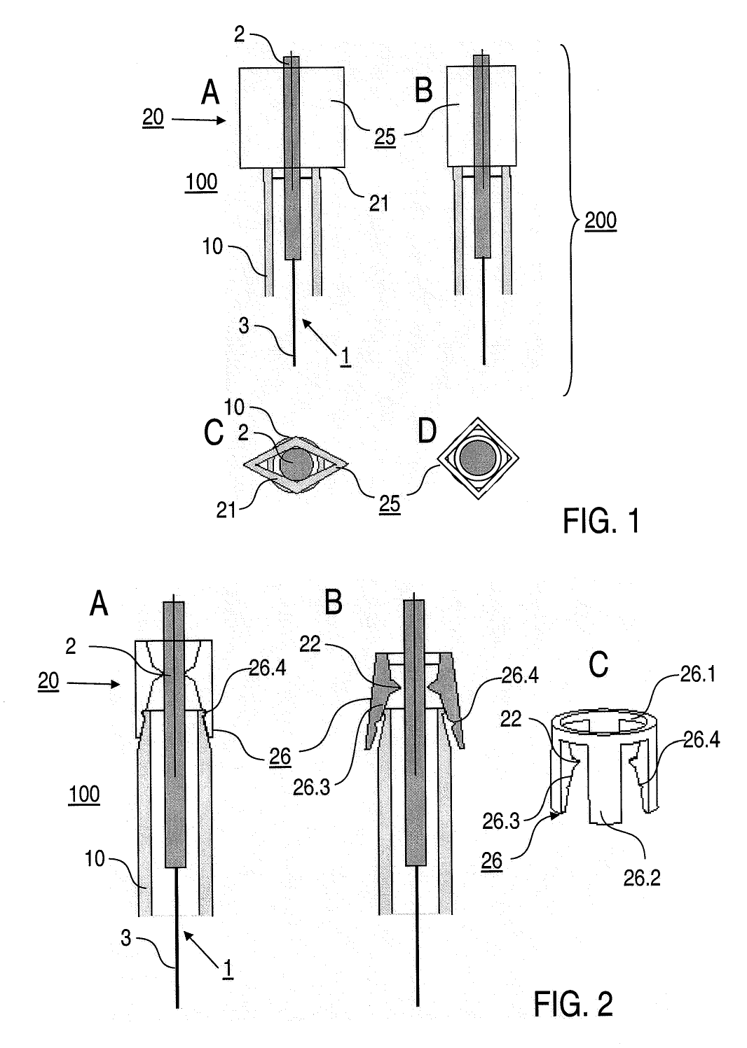

FIGS. 1 to 4 show different embodiments of guide devices according to the invention, and

FIG. 5 shows a conventional guide device for fixing an acupuncture needle to a guide tube (prior art).

The drawings illustrate examples of embodiments of the guide device 100 according to the invention, in each case with an acupuncture needle 1 in the clamped state or in the released state. The combination of the guide device 100 with the acupuncture needle 1 constitutes an acupuncture device 200 according to the invention. The acupuncture needle 1 is shown schematically with a needle handle 2 and a needle part 3 (shown in part). The implementation of the invention in practice is not limited to the acupuncture needles shown but rather is also possible with other acupuncture needles, in particular with other handle forms. Furthermore, the size ratios of the guide device 100 and the acupuncture needle 1 may be varied in practice.

According to FIG. 1, the guide device 100 comprises a guide tube 10 and a clamping device 20, which is arranged at the end of the guide tube 10. The guide tube 10 is made of plastic (e.g. polypropylene). The outer and inner diameters of the guide tube 10 are e.g. 4 mm and 3 mm. The clamping device 20 is formed by four spring clamping elements 21, which in the clamped state form a sleeve 25 with a cross section in the shape of a rhombus. The sleeve 25 with the spring clamping elements 21 is made of plastic (e.g. polypropylene). The length of the sleeve 25 in the axial direction (parallel to the axial extension of the guide tube 10) is preferably selected in the range from a few millimeters to 1.5 cm so that it can be operated manually using two fingers. The length of the sleeve 25 may in particular be selected as a function of the length of the needle handle.

The sleeve 25 is fixed to the end of the guide tube 10. For this, one or two adhesive bonds to the end of the guide tube 10 are provided for example on one of the spring clamping elements, while the other spring clamping elements are freely movable. As an alternative to the adhesive bonds, an integral connection may be provided between the spring element 21 and the end of the guide tube 10. According to another alternative, the entire sleeve 25 may be connected in one piece with the guide tube 10, provided that a mobility of at least one of the spring clamping elements with respect to the rest of the guide tube 10 is provided by the flexibility of the spring clamping elements 21. According to another alternative, the guide tube 10 may have a cross section in the shape of a rhombus over its entire length or over a partial region. In this case, the clamping device constitutes an extension on the guide tube. In this variant, the mobility of the clamping elements with respect to the guide tube is provided by the flexibility of the material of the guide tube at least in the region of the clamping device.

FIG. 1A shows the clamping device 20 in the clamped state, in which the needle handle 2 of the acupuncture needle 1 is clamped between the spring clamping elements 21. FIG. 1C shows this situation in plan view, with the diameter of the needle handle being shown on an enlarged scale for the sake of clarity. FIG. 1C shows that the sleeve 25 is arranged in such a way that the needle handle 2 is positioned axially and centrally in the guide tube 10. The release of the acupuncture needle 1 takes place in such a way that a radially inwardly acting force is exerted manually, e.g. by using two fingers, on the edges of the sleeve 25 which form the long diagonals of the rhombus cross-sectional shape. FIGS. 1B and 1C illustrate the clamping device 20 in the released state. Under the effect of the force acting along the long diagonals, the sleeve 25 is spread open so that in the interior a free space is formed which is larger than the diameter of the needle handle 2. Accordingly, the needle handle 2 is released from the clamping device 20. The acupuncture needle 1 slides through the guide tube 10 until the tip of the needle part 3 strikes the surface of the body part that is to be stimulated.

FIG. 2 shows a modified embodiment of the guide device 100 according to the invention, in which the clamping device 20 comprises a spreading sleeve 26 which has an inner profile and is placed onto the end of the guide tube 10. The spreading sleeve 26 has the shape of a crown with a ring 26.1, from which a plurality of sleeve blades 26.2 protrude axially. Formed on the inner side of the sleeve blades 26.2 are the profile clamping elements 22 which serve for releasably fixing the needle handle 2 of the acupuncture needle 1. Running from the free ends of the sleeve blades 26.2 towards the profile clamping elements 22 is an inclined run-up surface 26.3 with a groove 26.4 which cooperates with the end of the guide tube 10.

FIG. 2A shows the guide device 100 in the clamped state. The spreading sleeve 26 is seated on the guide tube 10, so that its end engages in the groove 26.4. In order to release the acupuncture needle 1, the spreading sleeve 26 is pushed axially along the guide tube 10 (FIG. 2B). The end of the guide tube 10 runs onto the inclined run-up surfaces 26.3, so that the sleeve blades 26.2 are spread open. Accordingly, the diameter of the seat formed by the profile clamping elements 22 increases, until the needle handle 2 is released.

FIG. 2C shows the spreading sleeve 26 by way of example with four sleeve blades 26.2 which are arranged radially at a distance from one another. In modified embodiments of the invention, fewer sleeve blades 26.2, e.g. two or three sleeve blades, may be provided. By way of example, in the case of three sleeve blades which are respectively arranged at angles of 120° apart, a particularly simple construction is obtained with a seat for the needle handle which is nevertheless stable and acts symmetrically on all sides. In further modifications, it is also possible for more than four sleeve blades to be provided.

In a manner differing from the configuration of the spreading sleeve 20 shown in FIG. 2, said spreading sleeve may have, instead of the sleeve blades, a closed circumferential wall with the profile clamping elements 22, the inclined run-up surfaces 26.3 and the groove 26.4 on the inner side.

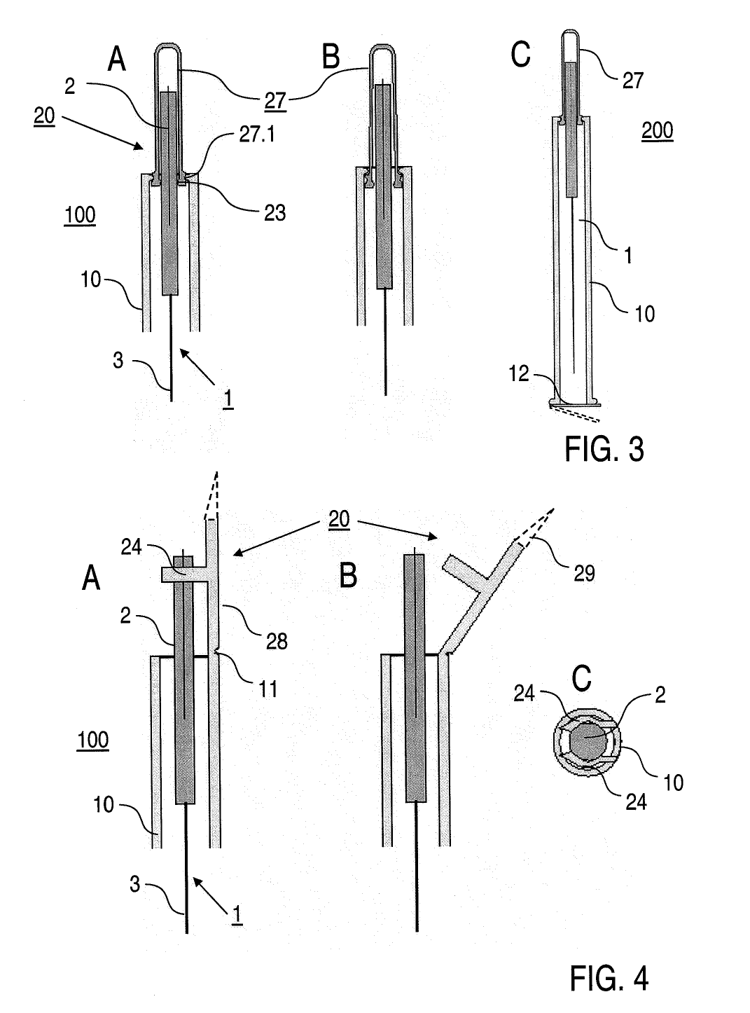

FIG. 3 illustrates a further embodiment of the guide device 100 according to the invention, in which the clamping device 20 is formed by a tubular sleeve 27 with an outer profile. The tubular sleeve 27 takes the form of a cap and has at one end an outer groove 27.1 which cooperates with the end of the guide tube 10. Unlike in the configuration shown in FIG. 2, the tubular sleeve 27 is not placed onto but rather is inserted into the end of the guide tube 10. Provided as clamping elements on the inner side of the tubular sleeve 27 are the profile clamping elements 23 which form a seat for the needle handle 2 of the acupuncture needle 1.

FIG. 3A shows the guide device 100 in the clamped state. The tubular sleeve 27 is compressed at its lower end by the guide tube 10 in such a way that the profile clamping elements 23 securely clamp the needle handle 2. The release of the acupuncture needle 1 takes place by exerting a force acting axially on the upper end of the tubular sleeve 27. The tubular sleeve 27 slides into the interior of the guide tube 10, whereby the lower end relaxes, so that the needle handle 2 is released (FIG. 3B).

The embodiment according to FIG. 3 has the advantage that the acupuncture device 200 can be provided as a bulk good, in which the acupuncture needle 1 is packaged in a sterile manner. To this end, as shown in FIG. 3C, the guide tube 10 is closed at the lower end by a layer-like cover 12, such as e.g. a film or paper, in particular so-called medical paper. The opposite end of the guide tube 10 is closed by the tubular sleeve 27. When the acupuncture needle 1 is released, the cover 12 can be broken through or preferably folded back, as illustrated by the cover shown in dashed line.

In FIG. 3, the tubular sleeve 27 is shown with a radially circumferential, closed wall in the form of a hollow cylinder (cap). According to an alternative embodiment of the invention, it may have wall openings or may comprise a group of sleeve clips which are connected at the upper end and at the lower end form the seat for the needle handle of the acupuncture needle.

FIG. 4 illustrates a further embodiment of the guide device 100 according to the invention, in which the clamping device 20 has gripper clamping elements 24 which are attached to a pivotable support 28. Each gripper clamping element 24 has a curved shape, so that a cylindrical seat for the needle handle 2 is formed between the gripper clamping elements 24. The support 28 is connected to the guide tube 10 via a film hinge. Located at the free end of the support 28 is a spike 29 which can be used as a tool when removing the acupuncture device from a packaging.

FIGS. 4A and 4C show the guide device 100 in the clamped state. The gripper clamping elements 24 on the support 28 aligned axially parallel to the guide tube 10 fix the needle handle axially and centrally in the guide tube 10. In order to release the acupuncture needle 1, the support 28 is pivoted as shown in FIG. 4B (released state).

Compared to the conventional technique, e.g. according to FIG. 5, the embodiment shown in FIG. 4 has the particular advantage that the gripper clamping elements 24 are made of a flexible material. The distance between the gripper clamping elements 24 can be selected to be so small that the needle handles of the smallest diameter which are used in practice (e.g. wire coil handles) can be securely clamped, and needle handles of larger diameter (e.g. plastic handles) can also be held.

The features of the invention which are disclosed in the above description, the drawings and the claims may be important both individually and in combination for implementing the invention in its various embodiments.

1. A guide device for an acupuncture needle, comprising:
a guide tube for accommodating the acupuncture needle, and
a clamping device for releasably fixing the acupuncture needle to the guide tube, wherein the clamping device has at least one clamping element which is movable with respect to the guide tube.

2. The guide device according to claim 1, in which the at least one clamping element is formed of an elastically deformable material.

3. The guide device according to claim 1, in which the at least one clamping element is provided for fixing at least part of the acupuncture needle axially and centrally in the guide tube.

4. The guide device according to claim 1, in which the clamping device is connected to the guide tube.

5. The guide device according to claim 4, in which the clamping device is integrally connected with the guide tube.

6. The guide device according to claim 4, in which the clamping device and the guide tube are releasably connected to one another.

7. The guide device according to claim 1, in which the at least one clamping element is part of a sleeve, inside which the acupuncture needle can be clamped.

8. The guide device according to claim 7, in which the sleeve is formed by spring clamping elements, between which the acupuncture needle can be clamped.

9. The guide device according to claim 8, in which the sleeve has a cross section in the shape of a rhombus.

10. The guide device according to claim 7, in which the sleeve has inner profile clamping elements, by means of which the acupuncture needle can be clamped.

11. The guide device according to claim 10, in which the sleeve is seated on one end of the guide tube and has an inner profile such that the sleeve can be narrowed by an axial movement relative to the guide tube, the profile clamping elements being part of the inner profile.

12. The guide device according to claim 10, in which the sleeve can be clamped in one end of the guide tube and has an outer profile such that the sleeve can be widened by an axial movement relative to the guide tube.

13. The guide device according to claim 7, in which the sleeve is closed at an end opposite the guide tube.

14. The guide device according to claim 1, in which the clamping elements comprise two gripper clamping elements, between which the acupuncture needle can be clamped.

15. The guide device according to claim 14, in which the gripper clamping elements are arranged on a radially pivotable support of the clamping device.

16. The guide device according to claim 15, in which the support is connected to the guide tube via a hinge.

17. The guide device according to claim 15, in which the support has a spike.

18. The guide device according to claim 1, in which the guide tube is closed at an end opposite the clamping device.

19. An acupuncture device, which comprises:
a guide device according to claim 1, and
an acupuncture needle.

20. The acupuncture device according to claim 19, in which the acupuncture needle has a needle handle which is connected to the clamping device of the guide device.

21. A method for releasing an acupuncture needle using an acupuncture device according to claim 19, comprising the step of deforming the clamping device of the guide device, so that the acupuncture needle is released.

22. The method according to claim 21, in which the clamping device is deformed by a radially acting force.

23. The method according to claim 21, in which the clamping device is deformed by an axially acting force.

24. The guide device according to claim 1, further comprising an acupuncture needle clamped by the clamping device.
